(19) United States
(12) Patent Application Publication
(10) Pub. No.: US 2024/0307219 A1
(43) Pub. Date: Sep. 19, 2024

(54) KNITTED THERMAL CIRCUIT SYSTEM

(71) Applicant: Baxter Medical Systems GmbH + Co. KG, Saalfeld (DE)

(72) Inventors: Jacqueline Elizabeth Burris, Indianapolis, IN (US); Matthew R. Knue, Sunman, IN (US); Timothy A. Lane, Greensburg, IN (US)

(73) Assignee: Baxter Medical Systems GmbH + Co. KG, Saalfeld (DE)

(21) Appl. No.: 18/606,727

(22) Filed: Mar. 15, 2024

Related U.S. Application Data
(60) Provisional application No. 63/452,845, filed on Mar. 17, 2023.

(51) Int. Cl. A61F 7/02; A61B 5/00; A61B 5/103; A61F 7/00; B32B 3/04; B32B 5/02; D04B 1/14; D04B 21/14

(52) U.S. Cl. CPC A61F 7/02; A61B 5/1036; A61B 5/447; A61B 5/6892; B32B 3/04; B32B 5/026; D04B 1/14; D04B 21/14; A61B 2505/05; A61F 2007/0071; A61F 2007/0093; A61F 2007/0253; A61F 2007/0257; A61F 2007/0288; B32B 2307/202; B32B 2307/206; B32B 2307/304; B32B 2307/7265; B32B 2535/00; D10B 2401/04; D10B 2401/16; D10B 2509/00

(57) ABSTRACT

A multi-layer pad for monitoring a patient includes first and second knitted fabric layers. The first knitted fabric layer includes first and second non-conductive bands and a first conductive strip that extend in a first direction. The first conductive strip is between the first and second non-conductive bands and is in contact with a power source. The second knitted fabric layer includes third and fourth non-conductive bands and a second conductive strip that extend in a second direction. The second conductive strip is between the third and fourth non-conductive bands and is in contact with the power source. A spacer is between the first and second knitted fabric layers. An impermeable cover layer encompasses the knitted fabric layers and the spacer. Upon application of force to the multi-layer pad, the first conductive strip is configured to move into close proximity with the second conductive strip and complete an electrical circuit.

### CROSS-REFERENCE TO RELATED APPLICATION

This application claims priority to and the benefit under 35 U.S.C. § 119(e) of U.S. Provisional Application No. 63/452,845, filed on Mar. 17, 2023, entitled “KNITTED THERMAL CIRCUIT SYSTEM,” the disclosure of which is hereby incorporated herein by reference in its entirety.

### FIELD OF THE DISCLOSURE

The present disclosure generally relates to a flexible multi-layer pad for supporting a patient, and more particularly to a flexible multi-layer pad for supporting a patient that shows areas of potential pressure sores.

### SUMMARY OF THE DISCLOSURE

According to one aspect of the present disclosure, a multi-layer pad assembly for use on a patient support includes a first knitted fabric layer and a second knitted fabric layer. The first knitted fabric layer includes a first plurality of conductive strips that extend in a first direction. The first plurality of conductive strips are in contact with a power source. A non-conductive band is disposed between adjacent conductive strips of the first plurality of conductive strips. The second knitted fabric layer includes a second plurality of conductive strips that extend in a second direction. The second plurality of conductive strips are in contact with the power source. A non-conductive band is disposed between adjacent conductive strips of the second plurality of conductive strips. The multi-layer pad also includes a spacer that is disposed between the first knitted fabric layer and the second knitted fabric layer. Upon application of a force to the first knitted fabric layer, the first plurality of strips is configured to move into close proximity with the second plurality of strips and complete an electrical circuit.

According to a second aspect of the present disclosure, a multi-layer pad for monitoring a patient includes a first knitted fabric layer and a second knitted fabric layer. The first knitted fabric layer includes first and second non-conductive bands that extend in a first direction and a first conductive strip that extends in the first direction and is disposed between the first and second non-conductive bands. The first conductive strip is in contact with a power source. The second knitted fabric layer includes third and fourth non-conductive bands that extend in a second direction and a second conductive strip that extends in the second direction and is disposed between the third and fourth non-conductive bands. The second conductive strip is in contact with the power source. The multi-layer pad also includes a spacer that is disposed between the first knitted fabric layer and the second knitted fabric layer. An impermeable cover layer encompasses the first knitted fabric layer, the second knitted fabric layer, and the spacer. Upon application of force to the multi-layer pad, the first conductive strip is configured to move into close proximity with the second conductive strip and complete an electrical circuit.

According to a third another aspect of the present disclosure, a multi-layer pad assembly for use on a patient support includes a first plurality of conductive strips that are spaced apart by first flexible non-conductive bands. The first plurality of conductive strips are in electrical communication with a power source. The multi-layer pad also includes a second plurality of conductive strips that are spaced apart by second flexible non-conductive bands. The second plurality of conductive strips are in electrical communication with the power source and overlay the first plurality of conductive strips to generally define a grid. The multi-layer pad further includes a compressible spacer that is disposed between the first plurality of conductive strips and the second plurality of conductive strips. Upon application of force to the multi-layer pad assembly, the compressible spacer is compressed and at least one of the first plurality of conductive strips comes into electrical communication with at least one of the second plurality of conductive strips to close an electrical circuit.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a top perspective view of a patient laying on a multi-layer pad of the present disclosure;

FIG. 2A is a top perspective exploded view of a multi-layer pad of the present disclosure;

FIG. 2B is a top perspective exploded view of another multi-layer pad of the present disclosure;

FIG. 3 is a top perspective cross-sectional view of a multi-layer pad of the present disclosure;

FIG. 4A is an enlarged cross-sectional view taken along a length of a multi-layer pad of the present disclosure;

FIG. 4B is an enlarged cross-sectional view taken along a width of the multi-layer pad of the present disclosure;

FIG. 4C is an enlarged cross-sectional view taken along a width of the multi-layer pad of the present disclosure;

FIG. 5 is a top perspective view of a patient laying on a multi-layer pad of the present disclosure;

FIG. 6 is a top perspective view of a multi-layer pad of the present disclosure illustrating circuit closures resulting from force applied by a weight of a patient;

FIG. 7 is a top perspective view of a multi-layer pad of the present disclosure illustrating an outline of a patient based on circuit closures provided as a result from force applied by a weight of the patient;

FIG. 8A is an outline of a small adult or child patient on a multi-layer pad illustrating associated pressure areas developed by the patient over time;

FIG. 8B is a cross-sectional view of the multi-layer pad taken at line VIII B-VIII B of FIG. 8A;

FIG. 9A is an outline of a small adult or child patient on a multi-layer pad illustrating associated pressure areas developed by the patient over time;

FIG. 9B is a cross-sectional view of the multi-layer pad taken at line VX B-VX B of FIG. 9A;

FIG. 10 is a block diagram illustrating a knitted thermal circuit system of the present disclosure; and

FIG. 11 is a flow diagram illustrating steps of a knitted thermal circuit system of the present disclosure in use.

### DETAILED DESCRIPTION

The present illustrated embodiments reside primarily in combinations of method steps and apparatus components related to a knitted thermal circuit system. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

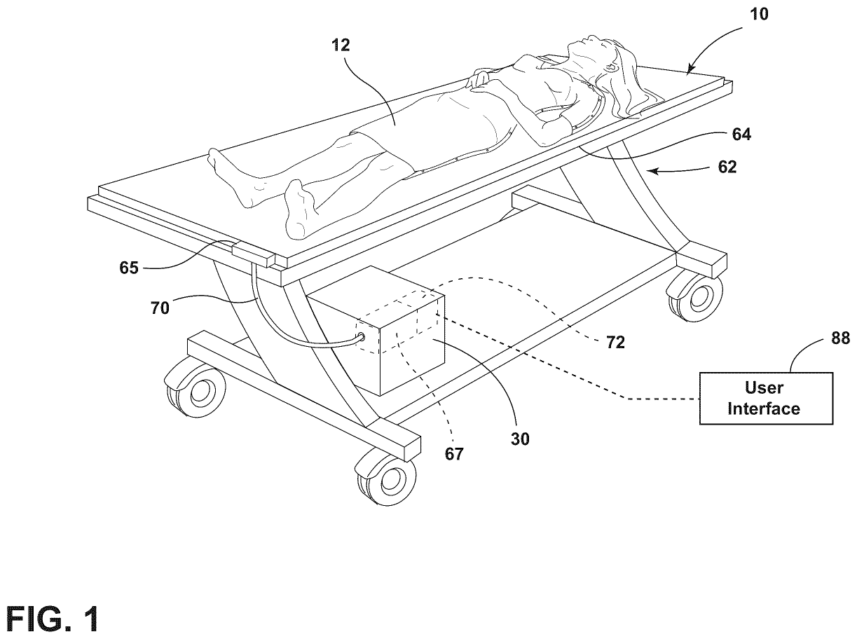

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

Referring to FIGS. 1-9B, reference numeral 10 generally designates a multi-layer pad for monitoring a patient 12 that includes a first knitted fabric layer 14 and a second knitted fabric layer 16. The first knitted fabric layer 14 includes non-conductive bands 18 that extend in a first direction 22 as well as conductive strips 24 that extend in the first direction 22 and are disposed between the non-conductive bands 18. The conductive strips 24 of the first knitted fabric layer 14 are in contact with a power source 30. The second knitted fabric layer 16 includes non-conductive bands 38 that extend in a second direction 42 as well as conductive strips 44 that extend in the second direction 42 and are disposed between the non-conductive bands 38. The conductive strips 44 of the second knitted fabric layer 16 are in contact with the power source 30. The multi-layer pad 10 also includes a spacer 50 that is disposed between the first knitted fabric layer 14 and the second knitted fabric layer 16. An impermeable cover layer 60 encompasses the first knitted layer 14, the second knitted fabric layer 16, and the spacer 50. Upon application of force to the multi-layer pad 10, the conductive strips 24 of the first knitted fabric layer 14 are configured to move into close proximity with the conductive strips 44 of the second knitted fabric layer 16 and complete an electrical circuit.

With reference again to FIG. 1, the patient 12 is illustrated positioned over the multi-layer pad 10 on a surgical table 62. Surgical tables, including the surgical table 62, are known to have cold and rigid support surfaces 64, which provide a stationary platform upon which a surgeon can perform surgical procedures. In addition, surgical suites are generally kept at a lower temperature, which is conducive to performing most surgeries. Often times, surgeries can last for several hours and, in rare events, may even last for days. Because of this, and the lack of movement by a patient, bedsores or other pressure related injuries can occur during surgery. Bedsores or pressure ulcers are types of injuries that generally break down skin and underlying tissue. Excessive pressure over a period of time disrupts regular blood flow through the skin. When sufficient blood supply is lacking, the affected skin becomes depleted of oxygen and nutrients and consequently begins to break down. Accordingly, excessive pressure over a long, or extended period of time increases the likelihood of developing bedsores or pressure ulcers. Pressure ulcers can range in severity from bruising to open wounds that can expose underlying tissue. In rare events, pressure ulcers can even lead to more severe sores that expose bone. There are many systems to aid in massaging the skin or otherwise moving a patient during a surgical procedure to alleviate the likelihood that pressure ulcers or sores will form. However, it may be difficult to discern where excessive pressure between a patient and a surgical table is occurring and, accordingly, where massaging or movement should occur. Consequently, a system that provides information related to excessive or prolonged pressure to particular portions of a patient would be useful.

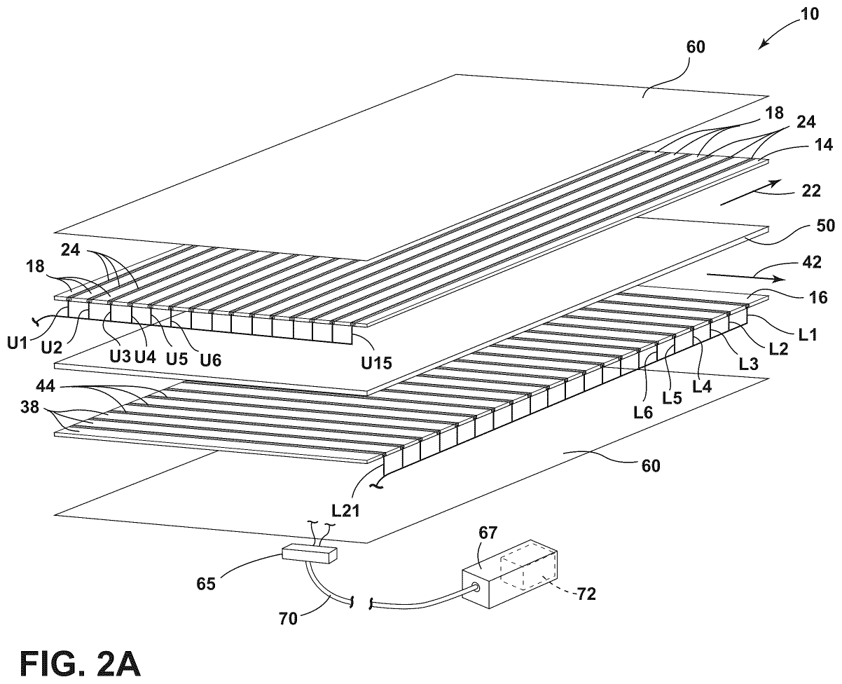

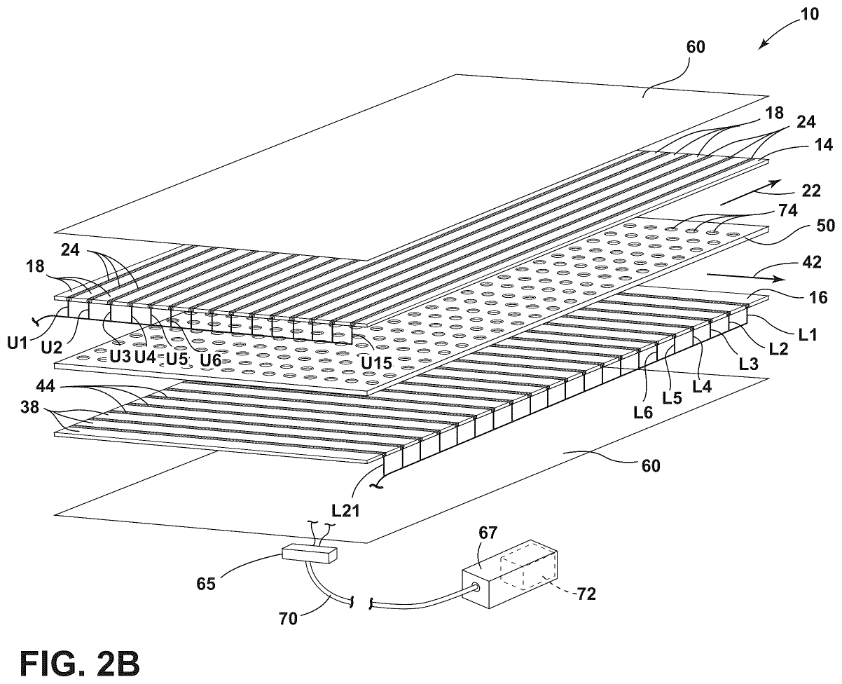

With reference now to FIGS. 1, 2A, and 2B, as previously noted, the multi-layer pad 10 includes several layers including the first knitted fabric layer 14 and the second knitted fabric layer 16. Notably, the first knitted fabric layer 14 and the second knitted fabric layer 16 may be identical in structure and dimensions. Alternatively, the first knitted fabric layer 14 may include a thicker construction than the second knitted fabric layer 16. In an alternative construction, the non-conductive bands 18 of the first knitted fabric layer 14 include a thickness or a width that differs from a thickness or a width of the non-conductive bands 38 of the second knitted fabric layer 16. The thickness of each non-conductive band 18 of the first knitted fabric layer 14 is sufficient to prevent any electrical communication between the conductive strips 24. The same is true of the non-conductive bands 38 of the second knitted fabric layer 16. The non-conductive bands 38 of the second knitted fabric layer 16 include a thickness sufficient to prevent electrical communication between adjacent conductive strips 44. With reference again to FIG. 2A, the conductive strips 24 of the first knitted fabric layer 14 and the conductive strips 44 of the second fabric layer 16 are arranged in a non-parallel orientation. As a result, a linear extent of the conductive strips 24 of the first knitted fabric layer 14 crosses a linear extent of the conductive strips 44 of the second knitted fabric layer 16. In some instances, as shown in FIG. 2A, the conductive strips 24 of the first knitted fabric layer 14 and the conductive strips 44 of the second knitted fabric layer 16 are arranged in a perpendicular orientation. However, it is also contemplated that the conductive strips 24 of the first knitted fabric layer 14 and the conductive strips 44 of the second knitted fabric layer 16 may be arranged at a variety of angles and need not necessarily be arranged at an orientation of 90° from one another. It will also be contemplated that the first and second knitted fabric layers 14, 16 may include thermally insulative properties that may assist with regulation of a core temperature and/or maintaining normothermia of the patient 12 during a surgical procure.

The spacer 50, as shown in FIG. 2A, includes a thickness sufficient to prohibit or prevent electrical communication between the conductive strips 24 of the first knitted fabric layer 14 and the conductive strips 44 of the of the second knitted fabric layer 16. It is generally contemplated that the spacer 50 may include a breathable material that has both stretchable and flexible characteristics. For example, a polyester spacer 50 that has a thickness of between 1 mm and 50 mm may be positioned between the first knitted fabric layer 14 and the second knitted fabric layer 16. It is contemplated that the material choice of the spacer 50 may be nearly any material that does not include conductive properties that would cause electrical communication between the conductive strips 24 of the first knitted fabric layer 14 and the conductive strips 44 of the second knitted fabric layer 16.

The impermeable cover layer 60 encompasses the first knitted layer 14 and the second knitted layer 16 and may be in the form of a bag into which the first knitted fabric layer 14, the second knitted fabric layer 16, and the spacer 50 are inserted and then sealed. Alternatively, the impermeable cover layer 60 made include a top layer and a bottom layer that are operably coupled about a periphery of the multi-layer pad 10. The top and bottom layers of the impermeable cover layer 60 may be connected via sonic welding, thermal welding, adhesion, etc. Regardless, the impermeable cover layer 60 is structured to provide flexibility such that the multi-layer pad 10 may be rolled up during non-use but will still maintain impermeable fluid-tight qualities when in use. In addition, the impermeable cover layer 60 maintains a close fit around the first knitted fabric layer 14 and the second knitted fabric layer 16, while keeping sufficient spacing between the first fabric layer 14 and the second fabric layer 16 about the periphery of the multi-layer pad 10 such that false readings of heightened pressure at the periphery do not occur.

With reference again to FIG. 2A, each of the conductive strips 24 of the first knitted fabric layer 14 are represented by lines U1, U2, . . . U15. Each of the conductive strips 44 of the second knitted fabric layer 16 are represented by lines L1, L2, . . . L21. Each line U1, U2, . . . U15 is in electrical communication with a wire harness 70 that is operably coupled with a junction box 65, which includes a controller 67 that includes a processor 72 that evaluates electrical signals that come from each of the conductive strips 24 (or the lines U1, U2, . . . U15) of the first knitted fabric layer 14. The controller 67 is also in electrical communication with each of the conductive strips 44 (or the lines L1, L2, . . . L21) of the second knitted fabric layer 16. The processor 72 is configured to process electrical signals from each of the conductive strips 24, 44, or the lines U1, U2, . . . U15, and the lines L1, L2, . . . L21, respectively, as discussed in further detail herein. The entire knitted thermal circuit system 100 may be powered by the power source 30 or a battery or other power source in communication with the controller 67.

With reference again to FIG. 2B, in an alternate construction, the spacer 50 includes a plurality of apertures 74 positioned at an intersection of each of the conductive strips 24 of the first knitted fabric layer 14 with the underlying conductive strips 44 of the second knitted fabric layer 16. As a result, as explained in further detail later, when pressure is applied to the impermeable cover layer 60, the conductive strips 24 of the first knitted fabric layer 14 are pressed into the spacer 50 such that at the plurality of apertures 74 of the spacer 50, where pressure has been applied, the conductive strips 24 of the first knitted fabric layer 14 and the conductive strips 44 of the second knitted fabric layer 16 abut or are in close proximity, which results in the conductive strips 24 of the first knitted fabric layer 14 coming into electrical communication with the conductive strips 44 of the second knitted fabric layer 16 and consequently, a closing of the electrical circuit of the knitted thermal circuit system 100 at those junctures.

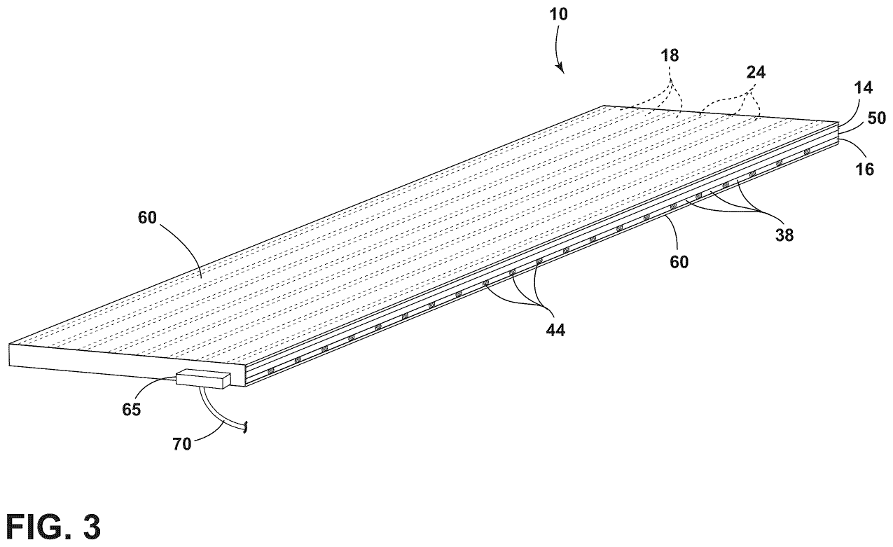

FIG. 3 illustrates a fully constructed multi-layer pad 10 of the knitted thermal circuit system 100 disclosed herein. As shown, the layers of the multi-layer pad 10 are in abutting contact with one another. The spacer 50 generally separates the first knitted fabric layer 14 from the second knitted fabric layer 16 so that the first knitted fabric layer 14 is not in electrical communication with the second knitted fabric layer 16. In addition, the impermeable cover layer 60 maintains the relationship between the first knitted fabric layer 14 and the second knitted fabric layer 16. It is contemplated that an adhesive may be utilized between the first and second knitted fabric layers 14, 16, and the spacer 50 to minimize the likelihood of internal wrinkling or unintentional spacing between the layers of the multi-layer pad 10. When in use, the multi-layer pad 10 has a generally planer construction that is adapted for use on a surgical table.

The multi-layer pad 10 has sufficient firmness and may include a density ranging from 1 lb/ft³ to 5 lbs/ft³. Is also contemplated that the spacer 50 of the multi-layer pad 10 may include a density or stiffness that is sufficient to space the first knitted fabric layer 14 from the second knitted fabric layer 16, but the spacer 50 will also be flexible or pliable enough to allow a force equivalent to the body weight of the patient 12 to move the conductive strips 24 of the first knitted fabric layer 14 into electrical communication with the conductive strips 44 of the second knitted fabric layer 16. An indentation load deflection (ILD) may range from 14 lbs-force to 44 lbs-force but may also range from 18 lbs-force to 24 lbs-force.

With regard to the impermeable cover layer 60, it is contemplated that the impermeable cover layer 60 may have sufficient stretch such that tension reduction on soft tissue or skin of the patient 12 is observed. As the impermeable cover layer 60 may include some degree of four-way stretch, tension on the skin of the patient 12 is reduced. Alternatively, the impermeable cover layer 60 may include a disposable stretchable and flexible outer layer such that the impermeable cover layer 60 itself may lack substantial stretchable or flexible characteristics.

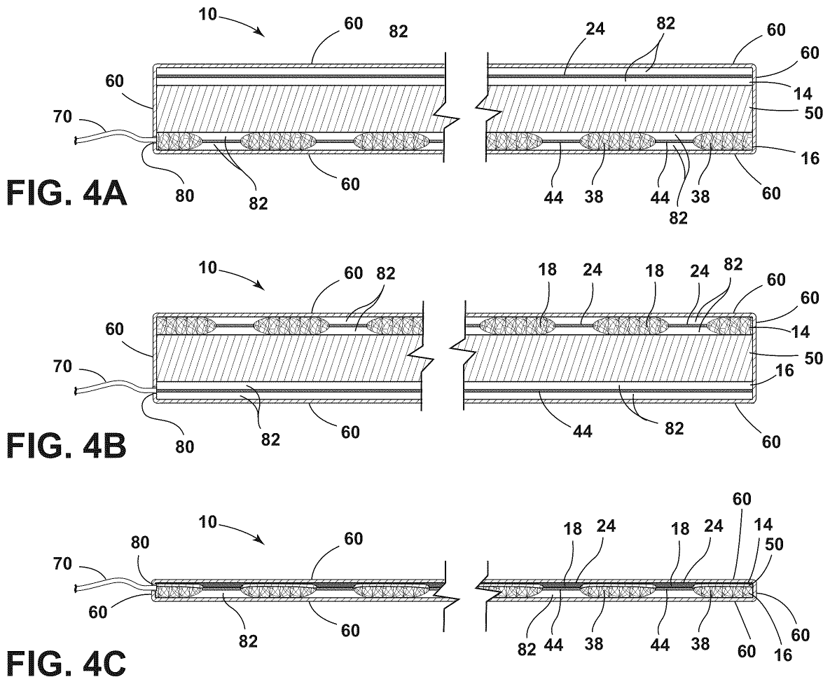

With reference now to FIGS. 4A-4C, a large cross-sectional view of the multi-layer pad 10 is provided. FIG. 4A represents a partial cross-sectional view taken along the length of the multi-layer pad 10. The impermeable cover layer 60 is disposed about an entirety of the periphery of the first knitted fabric layer 14, the spacer 50, and the second knitted fabric layer 16. Notably, a sealed opening 80 is provided that couples with the wire harness 70 that is in electrical communication with each of the conductive strips 24, 44. The wire harness 70 conveys data to the processor 72 which processes the data and provides output related to potential pressure areas on the patient 12, as discussed in further detail herein. The first knitted fabric layer 14 is disposed below the top layer of the impermeable cover layer 60. Notably, the thickness of the conductive strips 24 is less than that of the non-conductive bands 18 of the first knitted fabric layer 14. As a result, there may be some degree of open space 82 between the conductive strips 24 and the impermeable cover layer 60, as well as between the conductive strips 24 and the spacer 50.

The spacer 50 is positioned below the first knitted fabric layer 14 and is generally configured to abut both the first knitted fabric layer 14 and the second knitted fabric layer 16. As noted herein, the conductive strips 24, 44 may have a thickness that is less than the non-conductive bands 18, 38. Consequently open spaces 82 may exist between the conductive strips 24, 44 and the impermeable cover layer 60, and the conductive strips 24, 44 and the spacer 50. However, it is also contemplated that a thickness of the conductive strips 24 of the first knitted fabric layer 14 and the conductive strips 44 of the second knitted fabric layer 16 may be comparable or the same as the thickness of the non-conductive bands 18 of the first knitted fabric layer 14 and non-conductive bands 38 of the second knitted fabric layer 16, respectively, such that there are no open spaces 82. The thickness of the spacer 50 may depend on density, rigidity, and other physical characteristics of the material from which the spacer 50 is made. Regardless, when not in operation, the spacer 50 provides sufficient spacing between the first knitted fabric layer 14 and the second knitted fabric layer 16 to minimize or prevent any electrical communication between the conductive strips 24 of the first knitted fabric layer 14 and the conductive strips 44 of the second knitted fabric layer 16.

The non-conductive bands 18 of the first knitted fabric layer 14 and non-conductive bands 38 of the second knitted fabric layer 16, as well as the spacer 50, may be constructed from any of a number of polymeric materials that are sufficiently non-conductive. The conductive strips 24 of the first knitted fabric layer 14 and the conductive strips 44 of the second knitted fabric layer, however, may be constructed from any of a variety of conductive materials, including, but not limited to, metals such as silver, tin, copper, and nickel.

As can be seen at the bottom of the multi-layer pad 10, the conductive strips 44 of the second knitted fabric layer 16 are spaced intermittently along the multi-layer pad 10. It will be noted, however, that depending on the application, the conductive strips 44 of the second knitted fabric layer 16 may be more concentrated proximate to known load concentrations of the patient 12. For example, a higher density of conductive strips 44 may be proximate a head, shoulder, buttocks, and calf area of the patient 12 as those areas tend to have increased loading on a patient of average size.

FIG. 4B illustrates a partial cross-sectional view taken along a width of the multi-layer pad 10. Notably, the structure is very similar to that of FIG. 4A, but with the conductive strips 24 of the first knitted fabric layer 14 spaced intermittently along the top surface of the spacer 50. As with the conductive strips 44 of the second knitted fabric layer 16, the conductive strips 24 of the first knitted fabric layer 14 may be more concentrated at an inner region of a body of the multi-layer pad 10, as load concentrations proximate an interior portion of the multi-layer pad 10 are much likely to be higher than at the periphery of the multi-layer pad 10.

FIG. 4C illustrates the multi-layer pad 10 in a compressed state, which results from contact of the conductive strips 24 of the first knitted fabric layer 14 and the conductive strips 44 of the second knitted fabric layer 16 where the conductive strips 24 and the conductive strips 44 intersect. When the conductive strips 24 of the first knitted fabric layer 14 and the conductive strips 44 of the second knitted fabric layer 16 come in close contact or otherwise abut, the electrical circuit is closed and indicates that this area is a high pressure area for the patient 12.

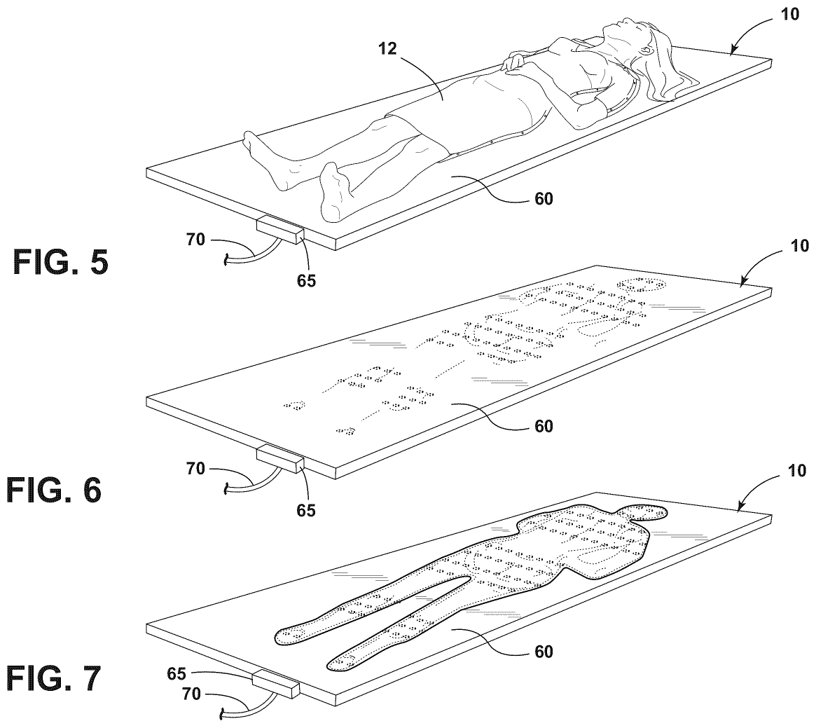

With reference now to FIGS. 5 and 6, the patient 12 is shown at rest prior to a surgical procedure. In this condition, the patient 12 applies some force to the multi-layer pad 10. This force results in the patient 12 pressing into a thickness of the multi-layer pad 10. As shown in FIG. 6, application of a force sufficient to close the electrical circuits at the head, shoulders, buttocks, calves and heels of the patient 12 indicates that those areas of the patient 12 are most likely to encourage sores or ulcers during the surgical procedure and should be monitored for such. It is generally contemplated that closure of the electrical circuits at portions of the body that penetrate into the multi-layer pad 10 also result in heating of the body at those areas (head, shoulders, buttocks, calves, and heels). It is also contemplated that the remainder of the multi-layer pad 10 may be configured so that heat is not admitted elsewhere. As a result, energy is saved and efficient use of energy by the multi-layer pad 10 for heating the patient 12 can be achieved.

FIG. 7 shows an outline of the patient 12 that can be determined based on the multi-layer pad 10, and an impression of the multi-layer pad 10 defined by the patient.

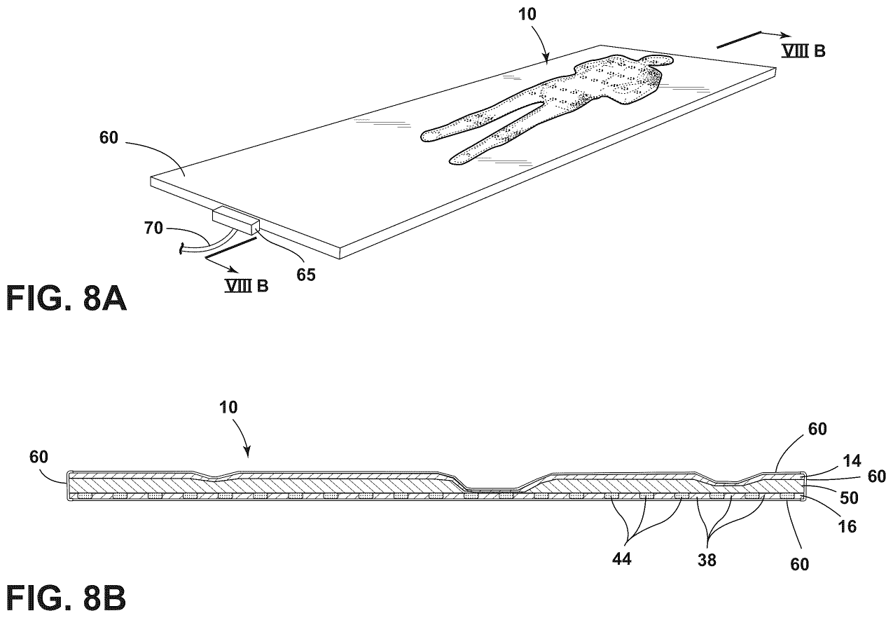

As shown in FIGS. 8A and 8B, a small, or child-sized, patient 12 will have minimal high-pressure zones as the small, or child-sized, patient 12 has smaller mass and, therefore, may not be able to create a force sufficient to cause one or more of the conductive strips 24 of the first knitted fabric layer 14 to electrically communicate with one of more of the conductive strips 44 of the second knitted fabric layer 16. However, it is also contemplated that even a very small patient may have sufficient weight to close at least some of the electrical circuits of the multi-layer pad 10 such that a caregiver will know where adjustment of a position of the patient or what areas of a patient's skin should be manually stimulated or massaged to prevent sores are ulcers.

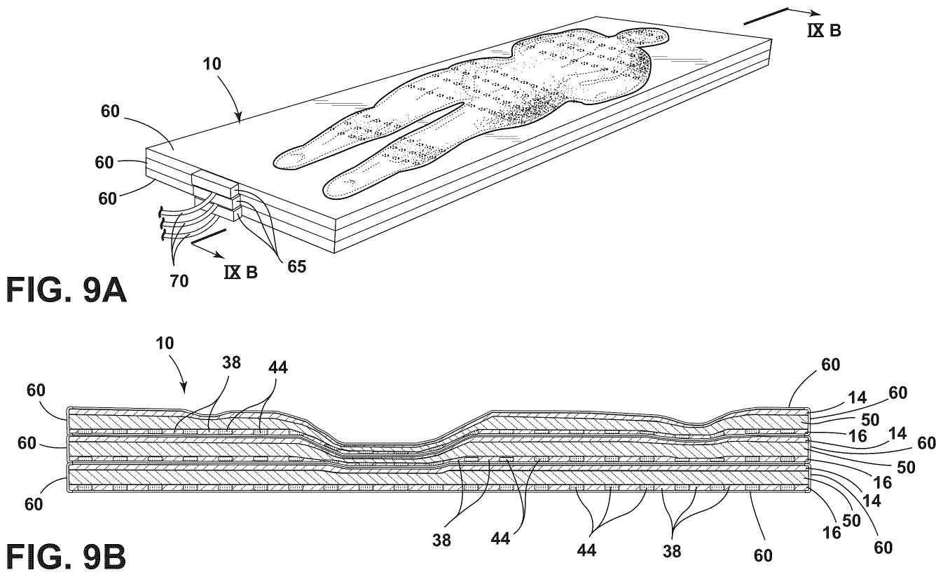

With Reference now to FIGS. 9A and 9B, for a larger patient, possibly an obese patient, several multi-layer pads 10 may be provided and stacked one upon the other. As shown in FIG. 9A, the patient may encompass a larger space and close more circuits than a person of smaller stature (FIG. 8A). This results in more pressure being applied through more multi-layer pads 10. As a larger patient will apply more pressure to the multi-layer pads 10, there may be more contact areas of the patient 12 to consider for pressure sores or ulcers.

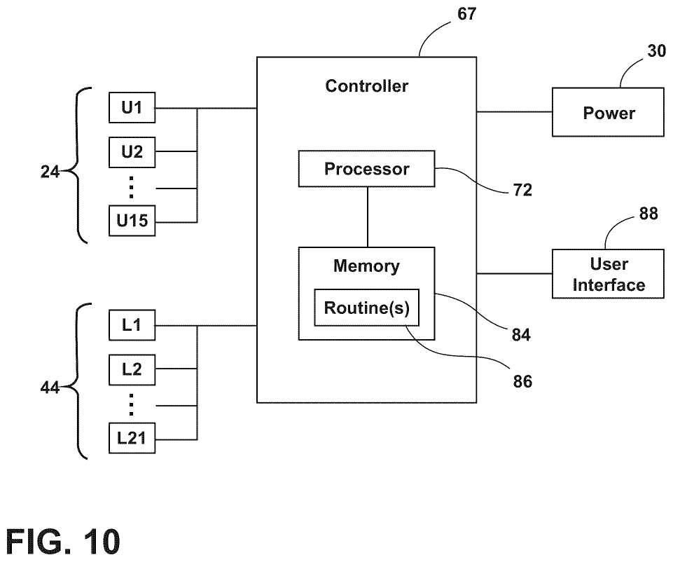

With reference now to FIG. 10, the controller 67 is operably coupled with a power source, such as the power source 30, which may be an external power source or a battery in close proximity to the knitted thermal circuit system 100. In addition, the controller is in electrical communication with each of the lines U1, U2, . . . U15 which represent each of the conductive strips 24 of the first knitted fabric layer 14. The controller 67 is configured to send a minimal amount of power through each of the lines U1, U2, . . . U15 and a capacitance or resistance of each of those lines U1, U2, . . . U15 is measured and monitored. The controller 67 is also in electrical communication with lines L1, L2, . . . L21, which represent the conductive strips 44 of the second knitted fabric layer 16. The controller 67 is also configured to send a minimal amount of power through each of the lines L1, L2, . . . L21 and a capacitance or resistance of each of those lines L1, L2, . . . L21 is measured and monitored. The controller 67 is operably coupled with the processor 72 that includes a memory 84 with routines 86. The processor 72 is configured to analyze data provided by lines U1, U2, . . . U15 as well as lines L1, L2, . . . L21. When certain lines intersect, and are forced into close proximity, or abutting contact with one another, the electrical circuit closes, and the resistance or capacitance in those lines changes. As a result, an indication that the length of the electrical circuit or circuits of several of the lines has changed or closed is received by the processor 72. The processor 72 then forwards this information to a user interface 88, which relays the information to the caregiver. The processor 72 may relay the information to the user interface 88 wirelessly via Bluetooth, WIFI, etc. The caregiver can then decide whether adjustment of the patient 12 on the surgical table 62 is appropriate, or if other treatment should be initiated to minimize the likelihood of pressure sores or ulcers.

For example, in one instance, when a patient 12 of average size is positioned on the multi-layer pad 10, the heels of the patient 12 may apply pressure at the intersection of the conductive strips 24, 44 at lines U3 and L19 and at lines U4 and L19, as well as at the intersection of lines U12 and L19, and also at lines U13 and L19. Once a predetermined threshold of time occurs at a predetermined loading, the user interface 88 may alert the caregiver to adjust one or both feet of the patient 12 or otherwise massage or stimulate blood flow to the heels of the feet. Of course, the user interface 88 may also provide alerts related to other body parts of the patient 12 once the time and force thresholds have been satisfied.

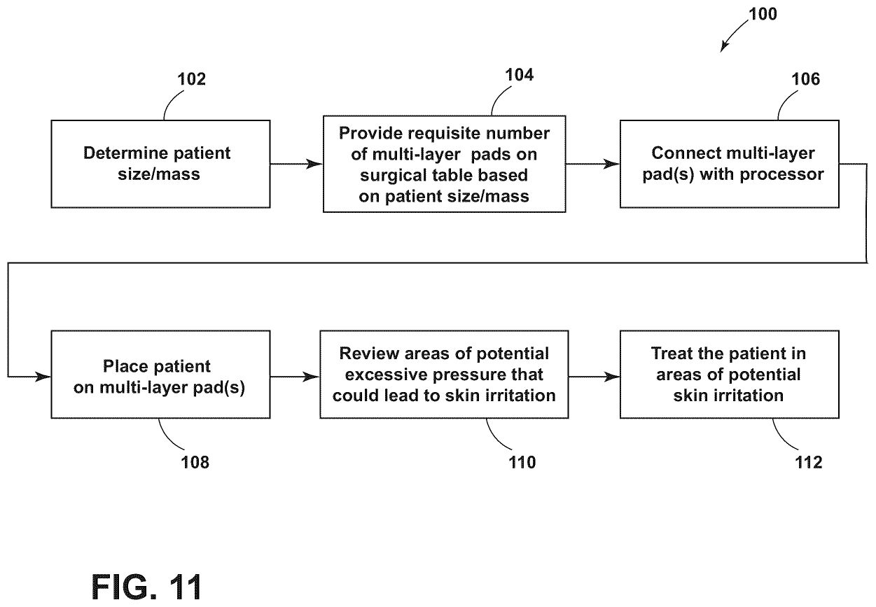

With reference now to FIG. 11, a flow diagram that illustrates steps of the knitted thermal circuit system 100 in use is shown. In a first step 102, a caregiver will determine a size and/or mass of the patient 12. Next, in step 104, based on the size and/or mass determination, the caregiver will provide a requisite number of the multi-layer pads 10 on the rigid support surface 64 of the surgical table 62. The multi-layer pad(s) 10 are then connected to the controller 67 via the wire harness 70. However, in some instances it is contemplated that the multi-layer pad(s) 10 may be wirelessly coupled to the controller 67. Moreover, the multi-layer pad(s) 10 may be inductively charged at the junction box 65, for example. As discussed in more detail above, several of the multi-layer pads 10 may be stacked one upon another to accommodate larger or heavier patients. In a next step 106, the multi-layer pad(s) 10 are connected to the processor 72 that will process electrical signals delivered from the conductive strips 24 of the first knitted fabric layer 14 and the conductive strips 44 of the second knitted fabric layer 16. The patient 12 is positioned on the multi-layer pads 10, at step 108. Next, in step 110, a caregiver or care provider team will review the data provided to the processor 72 by the multi-layer pad 10 to monitor areas of potential excessive pressure that could lead to skin irritation, including pressure ulcers or other sores on the skin of the patient 12. Then, in step 112, the caregiver or care team can treat the patient 12 as needed in those areas of potential excessive pressure that were identified in the step 110.

According to another aspect of the present disclosure, a multi-layer pad assembly for use on a patient support includes a first knitted fabric layer and a second knitted fabric layer. The first knitted layer includes a first plurality of conductive strips that extend in a first direction. The first plurality of conductive strips are in contact with a power source. A non-conductive band is disposed between adjacent conductive strips of the first plurality of conductive strips. The second knitted fabric layer includes a second plurality of conductive strips that extend in a second direction. The second plurality of conductive strips are in contact with the power source. A non-conductive band is disposed between adjacent conductive strips of the second plurality of conductive strips. The multi-layer pad also includes a spacer that is disposed between the first knitted fabric layer and the second knitted fabric layer. Upon application of a force to the first knitted fabric layer, the first plurality of strips is configured to move into close proximity with the second plurality of strips and complete an electrical circuit.

According to another aspect of the present disclosure, a first direction is substantially orthogonal to a second direction.

According to still another aspect of the present disclosure, a multi-layer pad assembly for use on a patient support includes an impermeable cover layer that encompasses a first knitted fabric layer, a second knitted fabric layer, and a spacer.

According to another aspect of the present disclosure, a spacer is configured to space a first knitted fabric layer from a second knitted fabric layer a distance to prohibit electrical communication between the first knitted fabric layer and the second knitted fabric layer.

According to yet another aspect of the present disclosure, a spacer includes a plurality of apertures juxtapositioned proximate overlapping sections of a first plurality of conductive strips and a second plurality of conductive strips.

According to still another aspect of the present disclosure, a non-conductive band of a first knitted fabric layer includes a thickness that prohibits adjacent conductive strips of a first plurality of conductive strips from electrical communication.

According to another aspect of the present disclosure, a first plurality of conductive strips and a second plurality of conductive strips are comprised of at least one of silver, copper, and nickel.

According to yet another aspect of the present disclosure, each of a first plurality of conductive strips and a second plurality of conductive strips is operably coupled with a relay connectable with a controller that monitors interactions of the first plurality of conductive strips with the second plurality of conductive strips.

According to still another aspect of the present disclosure, a pressure map associated with areas of excessive pressure applied by a patient is developed by a processor.

According to yet another aspect of the present disclosure, one or both of first and second knitted thermal fabric layers include thermally insulative properties.

According to another aspect of the present disclosure, a multi-layer pad for monitoring a patient includes a first knitted fabric layer and a second knitted fabric layer. The first knitted fabric layer includes first and second non-conductive bands that extend in a first direction and a first conductive strip that extends in the first direction and is disposed between the first and second non-conductive bands. The first conductive strip is in contact with a power source. The second knitted fabric layer includes third and fourth non-conductive bands that extend in a second direction and a second conductive strip that extends in the second direction and is disposed between the third and fourth non-conductive bands. The second conductive strip is in contact with the power source. The multi-layer pad also includes a spacer that is disposed between the first knitted fabric layer and the second knitted fabric layer. An impermeable cover layer encompasses the first knitted fabric layer, the second knitted fabric layer, and the spacer. Upon application of force to the multi-layer pad, the first conductive strip is configured to move into close proximity with the second conductive strip and complete an electrical circuit.

According to another aspect of the present disclosure, a spacer includes a plurality of apertures juxtapositioned proximate overlapping sections of a first conductive strip and a second conductive strip.

According to still another aspect of the present disclosure, at least one of a first conductive strip and a second conductive strip is comprised of at least one of silver, copper, and nickel.

According to another aspect of the present disclosure, each of a first conductive strip and a second conductive strip is operably coupled with a relay that is connectable with a controller that monitors interactions of the first conductive strip with the second conductive strip.

According to still another aspect of the present disclosure, a first conductive strip and a second conductive strip are in electrical communication with a relay via a wire harness disposed with an impermeable cover layer.

According to yet another aspect of the present disclosure, a first conductive strip is constructed from an elastic material.

According to another aspect of the present disclosure, a multi-layer pad assembly for use on a patient support includes a first plurality of conductive strips that are spaced apart by first flexible non-conductive bands. The first plurality of conductive strips are in electrical communication with a power source. The multi-layer pad also includes a second plurality of conductive strips that are spaced apart by second flexible non-conductive bands. The second plurality of conductive strips are in electrical communication with the power source and overlay the first plurality of conductive strips to generally define a grid. The multi-layer pad further includes a compressible spacer that is disposed between the first plurality of conductive strips and the second plurality of conductive strips. Upon application of force to the multi-layer pad assembly, the compressible spacer is compressed and at least one of the first plurality of conductive strips comes into electrical communication with at least one of the second plurality of conductive strips to close an electrical circuit.

According to another aspect of the present disclosure, a first plurality of conductive strips extend in a first direction and a second plurality of conductive strips extend in a second direction. The first direction is substantially orthogonal to the second direction.

According to still another aspect of the present disclosure, a multi-layer pad assembly for use on a patient support includes an impermeable cover layer that encompasses a first plurality of conductive strips, a second plurality of conductive strips, and a compressible spacer.

According to yet another aspect of the present disclosure, a compressible spacer includes a plurality of apertures juxtapositioned proximate overlapping sections of a first plurality of conductive strips and a second plurality of conductive strips.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A multi-layer pad assembly for use on a patient support, the multi-layer pad assembly comprising:
a first knitted fabric layer including:
a first plurality of conductive strips extending linearly in a first direction, the first plurality of conductive strips being in contact with a power source; and
a non-conductive band disposed between adjacent conductive strips of the first plurality of conductive strips;
a second knitted fabric layer including:
a second plurality of conductive strips extending linearly in a second direction, the second plurality of conductive strips being in contact with the power source; and
a non-conductive band disposed between adjacent conductive strips of the second plurality of conductive strips; and
a spacer disposed between the first knitted fabric layer and the second knitted fabric layer, wherein upon application of a force to the first knitted fabric layer, the first plurality of strips is configured to move into close proximity with the second plurality of strips and complete an electrical circuit, and wherein the spacer includes a plurality of apertures juxtapositioned proximate overlapping sections of the first plurality of conductive strips and the second plurality of conductive strips.

2. The multi-layer pad assembly of claim 1, wherein the first direction is substantially orthogonal to the second direction.

3. The multi-layer pad assembly of claim 2, further comprising:
an impermeable cover layer encompassing the first knitted fabric layer, the second knitted fabric layer, and the spacer.

4. The multi-layer pad assembly of claim 1, wherein the spacer is configured to space the first knitted fabric layer from the second knitted fabric layer a distance to prohibit electrical communication between the first knitted fabric layer and the second knitted fabric layer.

5. The multi-layer pad assembly of claim 4, wherein the non-conductive band of the first knitted fabric layer includes a thickness that prohibits adjacent conductive strips of the first plurality of conductive strips from electrical communication.

6. The multi-layer pad assembly of claim 5, wherein the first plurality of conductive strips and the second plurality of conductive strips are comprised of at least one of silver, copper, and nickel.

7. The multi-layer pad assembly of claim 1, wherein each of the first plurality of conductive strips and the second plurality of conductive strips is operably coupled with a relay connectable with a controller that monitors interactions of the first plurality of conductive strips with the second plurality of conductive strips.

8. The multi-layer pad assembly of claim 7, wherein a pressure map associated with areas of excessive pressure applied by a patient is developed by a processor.

9. The multi-layer pad assembly of claim 8, wherein at least one of the first and second knitted thermal fabric layers comprise thermally insulative properties.

10. A multi-layer pad for monitoring a patient, the multi-layer pad comprising:
a first knitted fabric layer including:
a first non-conductive band extending in a first direction;
a second non-conductive band extending in the first direction; and
a first conductive strip extending in the first direction and disposed between the first non-conductive band and the second non-conductive band, the first conductive strip being in contact with a power source, wherein the first conductive strip is at least partially constructed from an elastic material;
a second knitted fabric layer including:
a third non-conductive band extending in a second direction;
a fourth non-conductive band extending in the second direction; and
a second conductive strip extending in the second direction and disposed between the third non-conductive band and the fourth non-conductive band, the second conductive strip being in contact with the power source;
a spacer disposed between the first knitted fabric layer and the second knitted fabric layer; and
an impermeable cover layer encompassing the first knitted fabric layer, the second knitted fabric layer, and the spacer, wherein upon application of force to said multi-layer pad, the first conductive strip is configured to move into close proximity with the second conductive strip and complete an electrical circuit.

11. The multi-layer pad of claim 10, wherein the spacer is configured to space the first knitted fabric layer from the second knitted fabric layer a distance to prohibit electrical communication between the first knitted fabric layer and the second knitted fabric layer.

12. The multi-layer pad of claim 11, wherein the spacer includes a plurality of apertures juxtapositioned proximate overlapping sections of the first conductive strip and the second conductive strip.

13. The multi-layer pad of claim 12, wherein at least one of the first and second conductive strips is comprised of at least one of silver, copper, and nickel.

14. The multi-layer pad of claim 13, wherein each of the first conductive strip and the second conductive strip is operably coupled with a relay connectable with a controller that monitors interactions of the first conductive strip with the second conductive strip.

15. The multi-layer pad of claim 14, wherein the first conductive strip and the second conductive strip are in electrical communication with the relay via a wire harness disposed with the impermeable cover layer.

16. The multi-layer pad of claim 15, wherein a pressure map associated with areas of excessive pressure applied by said patient is developed by a processor.

17. The multi-layer pad of claim 16, wherein one or both of the first and second knitted thermal fabric layers comprise thermally insulative properties.

18. A multi-layer pad assembly for use on a patient support, the multi-layer pad assembly comprising:
a first plurality of conductive strips spaced apart by first flexible non-conductive bands, wherein the first plurality of conductive strips are in electrical communication with a power source;
a second plurality of conductive strips spaced apart by second flexible non-conductive bands, wherein the second plurality of conductive strips are in electrical communication with the power source, and wherein the second plurality of conductive strips overlay the first plurality of conductive strips to generally define a grid;
a compressible spacer disposed between the first plurality of conductive strips and the second plurality of conductive strips, wherein upon application of force to said multi-layer pad assembly, the compressible spacer is compressed and at least one of the first plurality of conductive strips comes into electrical communication with at least one of the second plurality of conductive strips to close an electrical circuit.

19. The multi-layer pad assembly of claim 18, further comprising:
an impermeable cover layer encompassing the first plurality of conductive strips, the second plurality of conductive strips, and the compressible spacer.

20. The multi-layer pad assembly of claim 19, wherein the compressible spacer includes a plurality of apertures juxtapositioned proximate overlapping sections of the first plurality of conductive strips and the second plurality of conductive strips.
